(19) United States
(12) Patent Application Publication
(10) Pub. No.: US 2025/0367347 A1
(43) Pub. Date: Dec. 4, 2025

(54) FIBRIN PATCH CONTAINING CORNEAL EPITHELIAL CELLS AND THE METHOD OF MANUFACTURING THEREOF

(71) Applicant: ACELLMED SPOLKA Z OGRANICZONA ODPOWIEDZIALNOSCIA, Zabrze (PL)

(72) Inventors: Rafal BULDAK, Zabrze (PL); Krzysztof PIETRYGA, Krakow (PL); Adam KONKA, Warszawa (PL); Katarzyna JESSE, Sosnowiec (PL); Joanna ZEMBALA-JOHN, Zbroslawice (PL)

(21) Appl. No.: 19/110,192

(22) PCT Filed: Sep. 11, 2023
(86) PCT No.: PCT/PL2023/050073

§ 371 Date: Mar. 10, 2025

(30) Foreign Application Priority Data
Sep. 12, 2022 (PL) P.442250

(51) Int. Cl. A61L 27/36; A61L 27/22; A61L 27/54; C12N 5/079

(52) U.S. Cl. CPC A61L 27/3666; A61L 27/225; A61L 27/54; C12N 5/0621; A61L 2300/64; A61L 2430/16; C12N 2500/84; C12N 2501/998; C12N 2501/999; C12N 2533/56

(57) ABSTRACT

The first object of the invention is a fibrin patch containing corneal limbal epithelial cells, characterized in that it contains a protein substrate, which is dried and rehydrated fibrin, a density of corneal limbal epithelial cells on the protein substrate is from 5,000 cells/cm2 to 70,000 cells/cm2, and the protein substrate does not contain a nutrient layer. The invention discloses also the method of the manufacturing of the patch.

The subject of the invention is a patch containing corneal limbal epithelial cells and a method of manufacturing a fibrin patch containing corneal limbal epithelial cells. The invention can be applied in the ophthalmic treatment.

The Polish validation of the European patent PL3509541T3 describes a method of producing a shaped lens subjected to the removal of cells from a source other than human donor corneal tissue. This lens has its major part with an anterior surface and a posterior surface where the lens is processed to break down immunogenic epitopes; at least part of the Bowman's membrane of the donor corneal tissue is retained as the anterior surface of the lens, and the posterior surface of said donor corneal tissue is shaped to give the major part of the lens a desired shape.

International application WO18015761A1 describes a biocompatible material for the delivery of a biological molecule to a target site, with the material consisting of a hydrogel matrix material, a divalent cationic-phosphate nanoparticle (in particular, calcium phosphate), and a biological molecule (in particular, nucleic acid) bound to the nanoparticle.

A method for forming three-dimensional tissue in vivo is known from the European patent EP2892577B1, involving the deposition of a composition containing cells or an extracellular matrix on a surface. In another instance, a method for forming a three-dimensional tissue in vivo is presented, which involves the deposition of at least one cell-containing composition, at least one extracellular matrix (ECM) composition, and at least one additional component on a surface in or on the subject.

From another European patent EP2975117B1, a method of obtaining cells derived from human corneal epithelial cells is known, which involves a process where cells of the nutrient layer are added to a culture vessel divided into the first and second chambers by a membrane having micropores that do not allow cells to pass, into the first chamber feeder layer cells are added and human corneal epithelial cells are added to the second chamber. Human corneal epithelial cells are cultured with a medium containing ROCK inhibitor and phosphodiesterase inhibitor, and then the said corneal epithelial cells are cultured in a second medium containing phosphodiesterase inhibitor, MAP kinase inhibitor, and TGF-B receptor inhibitor.

Another international application WO17118266A1 describes a method of producing an acellular cornea that includes the steps of subjecting an animal's cornea to a decellularization process that does not include the step of treating the cornea with protease, chelating agent, detergent, glycerol or a combination thereof. When the native cornea is treated, the native structure and conformation of the native cornea are preserved, while the immune response is reduced to such a level that the produced cornea can serve as a three-dimensional scaffold for host cells to grow on it after transplantation.

Another international application WO18015761A1 describes a biocompatible material for the delivery of a biological molecule to a target site, with the material consisting of a hydrogel matrix material, a divalent cation-phosphate nanoparticle (in particular, calcium phosphate), and a biological molecule (in particular, nucleic acid) bound to the nanoparticle, with the nanoparticle embedded in the hydrogel matrix material. This material, especially in three-dimensional form, can be applied to treat various diseases.

European patent application EP3823635A1 discloses compositions and a method for obtaining human limbal epithelial stem cells (LESC), supernatant from human corneal epithelial stem cells, or both, comprising: wetting and homogenization a corneal epithelial sample in a medium, drying the homogenized corneal epithelial sample until the edges of the sample adhere to the substrate, adding to the homogenized corneal epithelial sample a growth medium containing fetal bovine serum or an equivalent, without detaching the homogenized corneal epithelial sample from the substrate, with the amount of medium allowing at least part of the fragmented corneal epithelial sample to come into contact with air, culture of the fragmented corneal epithelial sample for one or more days, change of the growth medium to a medium containing human corneal growth supplement (HCGS) without fetal bovine serum, cell culture to grow human corneal epithelial stem cells, human corneal epithelial stem cell supernatant, or both.

Another European application EP3515600A1 discloses a method for manufacturing an organoid system, including: (i) seeding stem cells onto a surface, (ii) culturing stem cells from step (i) in situ to allow aggregation into multicellular aggregates containing stem cells, (iii) culturing multicellular stem cells containing aggregates from step (ii) in situ under conditions suitable for organoid development, where the arrangement of organoids is within a single focal plane, and where the surface can be covered regularly so that organoids are uniquely positioned in adjacent plates, and where the surface contains a bio functional hydrogel.

In yet another European application EP3522942A1, a method for forming a fibrin hydrogel composition is presented. This method includes the provision of a fibrin hydrogel or a precursor thereof, containing a fibrin hydrogel-forming salt. The concentration of the fibrin hydrogel-forming salt is greater than or equal to a threshold concentration for forming the fibrin hydrogel. The method further includes combining the fibrin hydrogel with a carrier material. The concentration of the carrier material is typically from 0.1 to about 50% by weight. The method further includes reducing the salt concentration below the threshold concentration to form the fibrin hydrogel.

Another European application EP3668967A1 discloses a method for the in vitro production of a patch of genetically modified cells on a fibrin substrate, where depositing feeder cells on the top surface of a fibrin substrate to obtain a fibrin substrate on which said feeder cells adhere; depositing and culturing genetically modified cells on a fibrin substrate to which said feeder cells adhere, said fibrin substrate being placed on fixed support so that the cells do not interact with the surface of said support, to obtain a patch of genetically modified cells adhering to said fibrin substrate; detaching a patch of genetically modified cells adhering to said fibrin substrate from the sheet-like substrate to obtain a patch of genetically modified cells on the fibrin substrate.

Another European application EP3967750A1 presents a method for producing a three-dimensional tissue construct, including a culture step involving growing cells in a culture medium containing fragmented extracellular matrix components, fibrin, and an aqueous medium.

On the other hand, the international application WO21167853A1 presents a method of manufacturing a scaffold for tissue engineering. The method includes applying at least one sheet of cells to a flexible scaffold, shaping the sheets to geometry, thus creating a tissue engineering scaffold. The preferred geometry is non-linear (i.e., not the essentially flat surface that a flat glass substrate can provide). The flexible scaffold is characterized by specific tensile strength, viscosity, strain, modulus, or any combination thereof.

The European application EP1451302A1 describes a method using stem cells. A small corneal biopsy—about 2mm2—is taken in the procedure. The biopsy is sent under certain conditions to the laboratory. In the laboratory, the limbal cells are multiplied in vitro to passage no. 2 in culture using a nutrient layer of fibroblasts. Once an adequate number of cells is reached, the cells are transferred to a fibrin carrier and cultured for 5-9 days until an epithelial monolayer is obtained (an average of 3.5% are stem cells; LESCs). The fibrin layer is obtained by casting Tisseel fibrin solution and salts (NaCl and HCl) into circular molds. A therapeutic product is formed from the cells and the scaffold. The therapeutic product is implanted into the patient's diseased eye approximately six months after the biopsy. Nevertheless, this method is characterized by several drawbacks. It is worth noting that cells cultured according to the cited solution require a nutrient layer composed of xenogeneic fibroblasts, which is not the method entirely preferred by the Committee for Advanced Therapies (CAT) of the European Medicines Agency (EMA).

The problem posed before the solution is to provide a patch for use in ophthalmic treatment, which would contain corneal limbal cells demonstrating therapeutic efficacy in vivo, and no nutrient layer would be necessary. Another problem would be to provide a method for obtaining such a patch.

The first object of the invention is a fibrin patch containing corneal limbal epithelial cells, characterized in that it contains a protein substrate, which is dried and rehydrated fibrin, the initial density of corneal limbal epithelial cells on the protein substrate is from 5,000 cells/cm² to 70,000 cells/cm², wherein the protein substrate does not comprise a nutrient layer.

Preferably, corneal limbal epithelial cells comprise at least 10% p63+ and Ki67+ cells.

In a further preferable embodiment of the invention, the initial density of the corneal limbal epithelial cells on the fibrin substrate is from 15,000 cells/cm² to 70,000 cells/cm², preferably from 19,000 cells/cm² to 70,000 cells/cm².

The second object of the invention is a method for manufacturing a fibrin patch, comprising of the following steps:
(a) preparing a mixture for producing a protein substrate, wherein the protein is fibrinogen,
(b) manufacturing the protein substrate,
(c) trimming of the scaffold for cell culture from the protein substrate,
(d) applying corneal limbal epithelial cells onto the scaffold,
(e) culturing the cells on the scaffold,
   characterized in that,
   in step (b), the mixture for making the protein substrate is poured into the bottom mold plate and allowed to gelate at 2-25° C. for 30 min to 48 h, then the protein substrate is dried to a water content of 5-95% by weight on air at 2-25° C. for 15 min to 48 h, then the substrate is rehydrated for 30 min to 2 h and in step (d), corneal limbal epithelial cells are seeded to the protein substrate to the initial density of 5,000 cells/cm² to 70,000 cells/cm², and in step (e) the corneal limbal epithelial cells are cultured on the scaffold prepared in step (c) until the surface of the scaffold is entirely overgrown by corneal cells.

In a preferable implementation of the invention, corneal limbal epithelial cells comprise at least 10% p63+ and Ki67+ cells.

In a further preferable embodiment of the invention, the initial density of the corneal limbal cells on the protein substrate is from 15,000 cells/cm² to 70,000 cells/cm², preferably from 19,000 cells/cm² to 70,000 cells/cm².

In yet another preferable embodiment of the invention, the mold comprises a top and a bottom plate, with the bottom plate comprising a cavity for pouring the mixture for obtaining the substrate.

In yet another preferable embodiment of the invention, cells are applied to the protein substrate using a pipette or a bioprinter.

In another preferable embodiment of the invention, the mixture for making the protein substrate comprises a 450 μl solution of fibrinogen at a concentration of 36 mg/ml to 44.5 mg/ml in PBS buffer, preferably also comprises aprotinin at a concentration of 500-1500 KIU/ml, a 25 μl solution of thrombin in PBS at a concentration of 50 IU/ml to 200 IU/ml and a 25 μl solution of aqueous calcium chloride at a concentration of 20 mM to 100 mM.

In another preferable embodiment of the invention, the mold is closed with a top plate.

In yet another preferable embodiment of the invention, a scaffold in the form of a disc is cut out in step (d) using a cutter.

In another preferable embodiment of the invention, the culture in step e) is carried out between 4 to 14 days, with the culture medium being changed over 2 to 3 days during the culture.

In another preferable embodiment of the invention, the culture in step e) is carried out in CNT medium with 10% (by weight) FBS content at 37° C. with a CO₂ concentration of 5% by volume. In another preferable embodiment of the invention, the culture in step e) is carried out in a mixture of culture media that includes CNT medium and DMEM medium in a volume ratio of 3:1-1/3:1, with the mixture comprising 0.5 mM sodium pyruvate, 5% by volume fetal bovine serum, 150 KIU/ml aprotinin, 0.5% by volume amino acid solution and 1% by volume antibiotic mixture comprising 100 U/ml penicillin, 100 μg/ml streptomycin, 10 μg/ml sparfloxacin at 37° C. with 5% by volume CO₂ and 95% humidity.

In yet another realization of the embodiment in step (b), the fibrin material is covered with 250 μl of 500 IU/ml thrombin solution, incubated at room temperature for 1 hour, then the thrombin is removed by washing it down with phosphate-buffered saline solution and dried for 15 minutes at room temperature at 50% humidity.

In another preferable embodiment, the substrate, after rehydration, is dried for 10 minutes at room temperature at 50% humidity.

In another preferable realization of the invention, cells on the protein substrate are applied with a pipette in such a way as the cells are in a concentrated suspension, the volume of which does not exceed 10 μl per cm2 of the scaffold.

In yet another preferable realization of the invention, cells are applied on the protein substrate using a bioprinter in such a way as the cells are in a concentrated suspension, the volume of which does not exceed 10 μl per cm2 of the scaffold.

In another preferable embodiment of the invention, after step (e), a scaffold with cells in the form of a disc is cut out using a cutter.

The patch, being the subject of the present invention, should be understood as a thin layer of transparent fibrin in the form of a hydrogel populated with corneal limbal cells and cultured in vitro until entirely covered by cells, which is then deposited on the patient's eye to regenerate the corneal epithelium and replenish the deficiency of corneal limbal stem cells.

The adequate density of corneal limbal stem cells on the scaffold ensures appropriate phenotype and viability of the cells propagated in vitro. The density of cells on the scaffold significantly increases the therapeutic efficacy of the preparation (patch according to the invention) as a result of obtaining a suitable growth niche for the transplanted cells. The density of cell deposition on the materials to be implanted seems to be of vital importance here (paracrine exchange of chemical signals between epithelial cells and corneal limbal stem cells). When the cell density is too high in the preparation, it may result in a limited growth surface for the transplanted cells. When the cell density is too low in the recipient site, it can activate apoptosis caused by anoikis or loss of cell-cell or cell-ECM adhesion [1]. In addition, according to the invention, the in vitro method of cell cultures, which are intended for transplantation, is carried out without the participation of xenogeneic cells at any stage of patch preparation-culture on fibrin alone.

Examples of the implementation of the invention are illustrated in the figure, where the figures show:

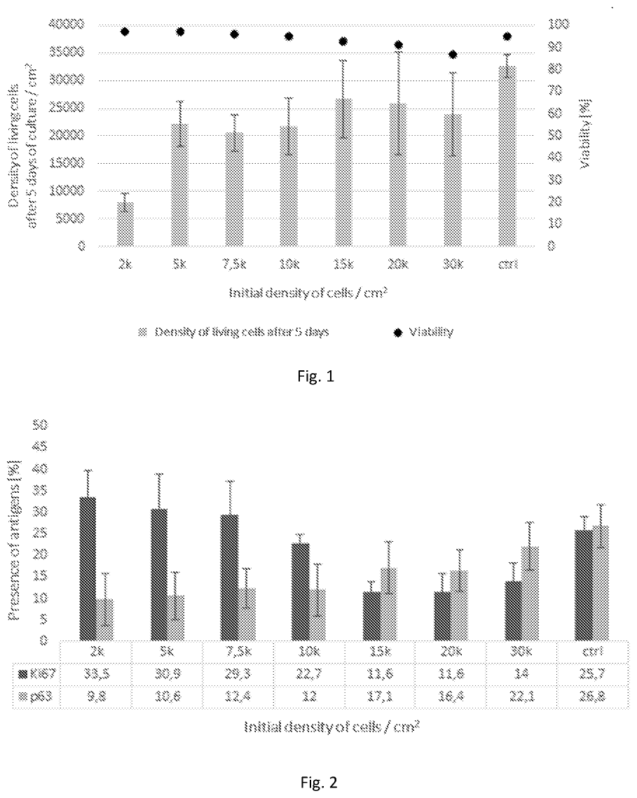

FIG. 1 Density of live cells after five days of in vitro culture on fibrin depending on the initial culture density (ctrl-control group on the surface of the culture plate at the density of 15,000 cells/cm2),

FIG. 2 Percentage distribution of selected corneal stem cell-specific markers (p63) and proliferative activity (Ki67) in relation to the initial density of culture on fibrin. (ctrl-control group on the surface of the culture plate at the density of 15,000 cells/cm2),

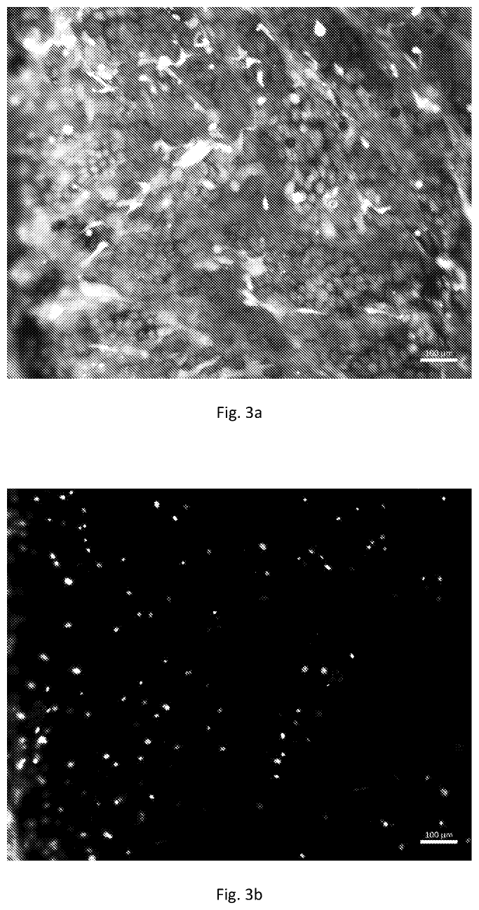

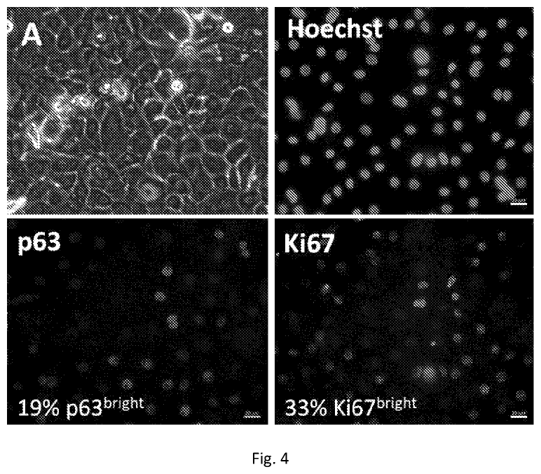

FIG. 3a-3b high viability of cells on the scaffold by Live-Dead staining method, where 3a—live cells, 3b—dead cells

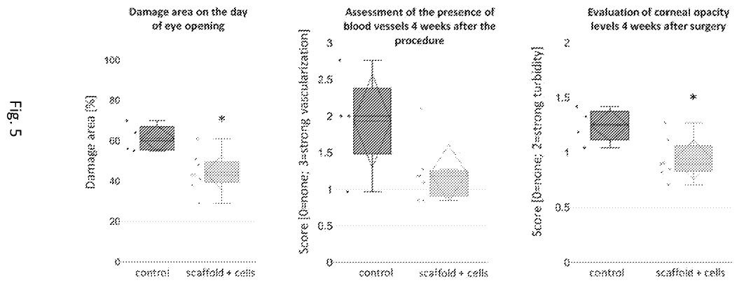

FIG. 4 Morphology of corneal limbal cells on the scaffold, staining of nuclei with Hoechst dye, and the presence of p63+ and Ki67+ cells on the scaffold (figure captions)—indicate the presence of corneal stem cells,

FIG. 5 Plots area presenting: eye damage vascularization and opacity scores for the test group with the patch against the control group. Statistical significance is marked with “*”.

### EXAMPLE 1

The fibrin substrate of the scaffold is produced in the form of a thin layer 0.5 mm thick by pouring the mixture for obtaining the protein substrate into the cavity space of the Teflon plate (bottom plate), which is part of the mold. The mold onto which the mixture is poured has the shape of a rectangular cavity (0.5 mm deep, 25 mm wide, and 50 mm long) and is closed from the top with a flat element (top plate). The mold has a two-element form, i.e., it contains a top plate for closing the mold and a bottom plate with a cavity for pouring the fibrinogen solution. The fibrinogen used is lyophilized fibrinogen from Baxter's TISSEEL Lyo kit dissolved in PBS (phosphate-buffered saline) to a final fibrinogen concentration of 40 mg/ml, containing thrombin at a concentration of 10 IU/ml (obtained from the TISSEEL kit and diluted in PBS) and calcium chloride at a concentration of 1 mM. The mixture for obtaining the protein substrate is prepared by dissolving 92 mg of fibrinogen in 2.068 ml of PBS under sterile conditions (the final concentration of fibrinogen is 44.5 mg/ml). Then 450 μl of this solution is taken and transferred to a tube containing 25 μl of thrombin (solution in PBS) at a concentration of 200 IU/ml and 25 μl of calcium chloride (solution in water) at a concentration of 20 mM. The composition of the mixture for obtaining the protein substrate may differ from the example cited above, for instance, in terms of the concentrations of the individual components. For example, the gelation time depends on the thrombin content.

The solution is mixed immediately, pipetted, and immediately, for example, in less than 1 min, poured into the mold. The mold for casting fibrin is sterilized in an autoclave at 121° C. Closing the mold with a top plate is not necessary to obtain the substrate; nevertheless, by doing so, we can obtain a uniform thickness and a flat surface to allow an even distribution of cells on the scaffold. The poured mixture is left to gelate at room temperature for 1 to 48 hours.

However, the present implementation example left it to gelate at 1 h. After removing the top mold, the material is dried to a water content less than or equal to 20 wt % in air at room temperature. The drying time can vary and depends on the mixture's initial water content. It can range from 12 to 48 hours. For example, for the mixture used according to this example, it is about 1 day. The gelled fibrin material is then rehydrated with water or PBS by pouring the liquid directly on the material and waiting at least 30 minutes until the hydrogel is swollen. However, for other compositions, the time required for rehydration may vary from 30 min to 2 h or more. From the fibrin material, discs of a diameter of 20 mm are punched with a cutter. They will become the scaffold for the cells. All operations on the material are performed under sterile conditions using sterile reagents.

The fibrin patch is produced by applying corneal stroma epithelial cells isolated from harvested corneal limbal epithelial fragments and cultured in CnT-PR medium from CELLnTEC being on passage 2. The culture yields 2 million viable cells, at least 3.5% of which are corneal limbal stem cells. By cytometric method or fluorescence microscopy, phenotypic characteristics are confirmed. Application is carried out by manually introducing the cell suspension onto the fibrin material so that the initial density of cells per 1cm2 of the scaffold surface is between 5000 cells/cm2 (cells per cm2) and 30000 cells/cm2. Cell culture on fibrin material is carried out until the surface is entirely overgrown by cells, usually for a period of 2-5 days. The degree of cell coverage of the surface is assessed daily using a phase contrast microscope. The culture medium (CNT medium + 10% FBS by volume) is replaced every 2nd day. Cell culture is carried out under standard conditions in an incubator providing 37° C., 5% CO2, and 95% humidity. The way the cell culture is carried out on the scaffold also makes it possible to cut it out with the help of a cutter after its completion.

The aforementioned initial density of cultured cells on the fibrin scaffold created in this way results in a phenotype characteristic of cells showing therapeutic efficacy in vivo. These conditions result in a scaffold with a cell density of more than 20000 cells/cm2 and high viability, as illustrated in FIG. 1. At the same time, a phenotype characterized by the presence of p63+ and Ki67+ cells in culture at a level of more than 10% is maintained throughout this range as shown in FIG. 2. It is preferable to apply cells in the form of a concentrated suspension, the volume of which does not exceed 10 μl per cm2 of the scaffold.

All operations are carried out under sterile conditions. A sterilized instrument and sterile reagents are used, or they are subjected to thermal sterilization or filtration. Work with fibrin material and cells is carried out under sterile conditions.

### EXAMPLE 2

The patch was produced similarly to the example 1. The fibrin substrate for the scaffold is produced in the form of a thin layer 0.7 mm thick by pouring the mixture for obtaining the protein substrate into the cavity space formed by the Teflon plate and the metal ring, which is the lower element of the mold. The Teflon element (plate) contains 200 μm circular cavities with a diameter of 0.5 mm in a rectangular grid arrangement with a lattice size of 1 mm×1 mm. The mold is closed from the top with a flat Teflon element. As a material, lyophilized fibrinogen from Baxter's TISSEEL Lyo kit dissolved in PBS (phosphate-buffered saline) to a final fibrinogen concentration of 36 mg/ml, additionally containing thrombin at a concentration of 2.5 IU/ml (obtained from the TISSEEL kit and diluted in PBS), calcium chloride at a concentration of 5 mM and aprotinin at a concentration of 1000 KIU/ml. The mixture for obtaining the protein substrate is prepared by dissolving 91 mg of fibrinogen in 1 ml of PBS containing aprotinin 2506.9 KIU/ml under sterile conditions. Then 500 μl of this mixture is taken and diluted with 638 μl of PBS. Then 450 μl of this solution is taken and transferred to a tube containing 25 μl of thrombin (solution in PBS) at 50 IU/ml and 25 μl of calcium chloride (solution in water) at 100 mM. The composition of the mixture for obtaining the protein substrate may differ from the example cited above, for instance, in terms of the concentrations of the individual components. The gelation time, for example, depends on the thrombin content. The solution is mixed immediately by pipetting and, for example, in less than 1 minute, is immediately poured into the mold. The mold is prepared by previously sterilizing it in an autoclave at 121° C. Immediately, the upper mold is closed. The poured mixture is left to gelate at a temperature of 2° C. to 25° C., and preferably at room temperature, for 30 minutes. After removing the upper mold, the material is covered with 250 μl of 500 IU/mL thrombin solution and incubated at room temperature for 1 hour. The thrombin is then removed by rinsing the scaffold with PBS solution. The fibrin material is dried for 15 minutes at a temperature range of 2° C. to 25° C., and preferably at room temperature, in humidity of 50%. Drying is carried out to a water content of 5% to 95% by weight, preferably up to 80% by weight. The gelled fibrin material is sealed in an airtight container and stored at 4° C. for 3 days. The fibrin material is prepared for cell seeding by cutting a 20 mm diameter disc, placing it in a culture dish, and pouring 2 ml of CNT culture medium with 5% FBS and antibiotics (100 U/ml penicillin, 100 μg/ml streptomycin) at 37° C. for 1 hour-a rehydration process. All operations on the material are performed under sterile conditions using sterile reagents.

Fibrin patch is produced by applying corneal limbal epithelial cells isolated from harvested corneal limbal epithelial fragments and cultured in CNT medium (CELLnTEC), with the addition of 5% by volume FBS (Fetal Bovine Serum-Fetal Bovine Serum) and 1% by volume antibiotics (final concentrations: 100 U/ml penicillin, 100 μg/ml streptomycin, 10 μg/ml sparfloxacin) being at passage 2. The said cell culture is performed under standard conditions in an incubator providing 37° C., 5% CO2, and 95% humidity. The cultures are characterized by the presence of p63+ and Ki67+ cells at min. 10%.

Corneal limbal epithelial cells are applied on a hydrated scaffold, which is additionally subjected to a 10-minute drying process at room temperature and 50% humidity. Application of the cells is carried out by manually dispensing the cell suspension using a pipette, or, for example, through a bioprinter, onto the fibrin material in such a way as the initial density of cells per cm2 of the scaffold surface is between 19,000 and 70,000 cells, preferably 68,789cells. This density is obtained by applying 12 μl of a cell suspension with a density of 18,000,000 cells/ml. It is preferable to apply the cells, using a pipette or a bioprinter, as a concentrated suspension, the volume of which does not exceed 10 μl per cm2 of the scaffold. The culture of corneal limbal epithelial cells on fibrin material is carried out until the surface is entirely overgrown and the cells form a compact epithelial layer, usually for 4-14 days. The degree of cell coverage of the surface is assessed using a phase contrast microscope. Cell culture on fibrin material is carried out in a mixture of CNT (CELLnTEC) and DMEM (Dulbecco's Modified Eagle Medium) culture media in a ratio of 3:1-1/3:1, and preferably 1:1, with the addition of 0.5% by volume of sodium pyruvate (final concentration of 0.5 mM), 0.5% by volume amino acid solution (non-essential amino acids, Gibco), 5% by volume FBS, 1% by volume antibiotics (final concentration of 100 U/ml penicillin, 100 μg/ml streptomycin, 10 μg/ml sparfloxacin) and the addition of 150 KIU/ml aprotinin. The culture media mixture is replaced every 2-3 days. Cell culture is carried out under standard conditions in an incubator providing 37° C., 5% CO2, and 95% humidity. The cell culture method on the scaffold also allows to cut out a fragment of it with the help of a cutter after its completion.

The aforementioned initial density of cultured cells on the fibrin scaffold created in this way results in a phenotype characteristic of cells showing therapeutic efficacy in vivo. These conditions result in a scaffold with a cell density of more than 100,000 cells/cm2 and high viability (more than 80%) as illustrated in FIG. 3a-3b, which shows cells stained by the Live-Dead staining method: 3a—live cells, 3b—dead cells. At the same time, the phenotype is preserved, characterized by the presence of p63+ and Ki67+ cells in the culture at the level of more than 10% as depicted in FIG. 4.

All operations are carried out while maintaining sterile conditions. Sterilized instruments and sterile reagents are used or subjected to thermal sterilization or filtering. Work on fibrin material and application of cells is carried out under sterile conditions.

### EXAMPLE 3 THERAPY WITH FIBRIN PATCH

The fibrin patch produced according to Example 2 was applied to a pig model with a damaged corneal limbus and corneal epithelium. The damage procedure consisted of exposing the eye to 20% ethyl alcohol, followed by mechanical removal of the epithelium with surgical instruments. The fibrin patch was placed under the conjunctival fold and fixed with 3 sutures, and then suturing of the 3rd eyelid and tarsorrhaphy was performed. After 5 days of observation, the eye was opened, and further observations were made for one month. The opacity, the presence of vessels, and the rate of corneal epithelialization were evaluated.

There was a statistically significant acceleration of the corneal epithelialization rate 5 days after treatment for the test group compared to the control group (FIG. 5). After 30 days of treatment, the level of vascularization and corneal opacity were significantly reduced for the test group compared to the control, which may indicate a positive outcome of the therapy (FIG. 5).

### LITERATURE

1. Ningning He, Yang Xu, Wei Du, Xin Qi, Lu Liang, Yuebing Wang, Guowei Feng, Yan Fan, Zhongchao Han, Deling Kong, Zhen Cheng, Joseph C. Wu, Zuoxiang He & Zongjin Li. Extracellular matrix can recover the downregulation of adhesion molecules after cell detachment and enhance endothelial cell engraftment, Sci. Rep. 5 (2015) 10902.
2. Zvibel I, Smets F, Soriano H. Anoikis: roadblock to cell transplantation? Cell Transplant 2002;11(7):621-30.
3. Flusberg, D. A.; Numaguchi, Y.; Ingber, D. E. Cooperative control of Akt phosphorylation, bcl-2 expression, and apoptosis by cytoskeletal microfilaments and microtubules in capillary endothelial cells. Mol. Biol. Cell 12:3087-3094; 2001.
4. Robey T E, Saiget M K, Reinecke H, Murry C E. Systems approaches to preventing transplanted cell death in cardiac repair. J Mol Cell Cardiol. 2008 October;45(4):567-81. doi: 10.1016/j.yjmcc.2008.03.009. Epub 2008 Mar. 19.

1. A fibrin patch containing corneal limbal epithelial cells, characterized in that it comprises a protein substrate, which is dried and rehydrated fibrin, the initial density of corneal limbal epithelial cells on the protein substrate is from 5,000 cells/cm2 to 70,000 cells/cm², and the protein substrate does not comprise a feeder layer.

2. The patch according to the claim 1, characterized in that corneal limbal epithelial cells comprise at least 10% p63+ and Ki67+ cells.

3. The patch according the claim 1 or 2, characterized in that the initial density of the corneal limbal epithelial cells on the fibrin substrate is from 15,000 cells/cm² to 70,000 cells/cm², preferably from 19,000 cells/cm² to 70,000 cells/cm².

4. A method for manufacturing the fibrin patch, comprising following steps:
(a) preparing a mixture for producing a protein substrate, wherein the protein is fibrinogen,
(b) manufacturing the protein substrate,
(c) trimming of the scaffold from the protein substrate,
(d) applying corneal limbal epithelial cells onto the scaffold,
(e) culturing the cells on the scaffold,
and characterized in that,
in step (b), the mixture for making the protein substrate is poured into the bottom mold plate and allowed to gelate at 2-25° C. for 30 min to 48 h, then the protein substrate is dried to a water content of 5-95% by weight on air at 2-25° C. for 15 min to 48 h, then the substrate is rehydrated for 30 min to 2 h and in step (d), corneal limbal epithelial cells are seeded to the protein substrate to an initial density of 5,000 cells/cm2 to 70,000 cells/cm2, and in step (e) the corneal limbal epithelial cells are cultured on the scaffold prepared in step (c) until the surface of the scaffold is entirely overgrown by corneal limbal cells.

5. The method according the claim 4, characterized in that corneal limbal epithelial cells comprise at least 10% of p63+ and Ki67+ cells.

6. The method according the claim 4, characterized in that the initial density of the corneal limbal cells on the protein substrate is from 15,000 cells/cm² to 70,000 cells/cm², preferably from 19,000 cells/cm² to 70,000 cells/cm².

7. The method according the claim 4 or 7, characterized in that the mold comprises a top and a bottom plate, where the bottom plate has a cavity for pouring the mixture for obtaining the substrate.

8. The method according to the claim 4, characterized in that the cells are applied to the protein substrate using a pipette or a bioprinter.

9. The method according to the claim 4, characterized in that the mixture for making the protein substrate comprises a 450 μl solution of fibrinogen at a concentration of 36 mg/ml to 44.5 mg/ml in PBS buffer, a 25 μl solution of thrombin in PBS at a concentration of 50 IU/ml to 200 IU/ml and a 25 μl solution of aqueous calcium chloride at a concentration of 20 mM to 100 mM, preferably also comprises aprotinin.

10. The method according to the claims 4 to 7, characterized in that the mold is closed by the top plate.

11. The method according to the claim 4, characterized in that in step d) the scaffold in the form of a disc is cut out using a cutter.

12. The method according the claim 4, 5 or 6, characterized in that the culture in step e) is carried out between 4 to 14 days, with the culture medium being changed over 2 to 3 days.

13. The method according to the claim 4 or 12, characterized in that the culture in step e) is carried out in CNT medium with 10% (by weight) FBS content at 37° C. with a CO₂ concentration of 5% by volume and 95% humidity.

14. The method according to the claim 4 or 12, characterized in that the culture in step e) is carried out in a mixture of culture media that comprises CNT medium and DMEM medium in a volume ratio of 3:1-1/3:1, preferably 1:1, with the mixture containing 0.5 mM sodium pyruvate, 5% by volume fetal bovine serum, 150 KIU/ml aprotinin, 0.5% by volume amino acid solution and 1% by volume antibiotic mixture containing 100 U/ml penicillin, 100 μg/ml streptomycin, 10 μg/ml sparfloxacin, at 37° C. with 5% by volume CO₂ and 95% humidity.

15. The method according the claim 4, characterized in that after gelation in step b) the fibrin material is covered with 250 μl of 500 IU/ml thrombin solution, incubated at room temperature for 1 hour, then the thrombin is removed by washing it down with phosphate-buffered saline solution and dried for 15 minutes at room temperature and at 50% humidity.

16. The method according to the claim 4, characterized in that the substrate, after rehydration, is dried for 10 minutes at room temperature at 50% humidity.

17. The method according to the claim 4, characterized in that in step d) the scaffold in the form of a disc is being cut our using a cutter.

18. The method according to the claim 4 or 8, characterized in that cells on the protein substrate are applied using a pipette in such a way as the cells are in a concentrated suspension, the volume of which does not exceed 10 μl per cm² of the scaffold.

19. The method according to the claim 4 or 8, characterized in that cells on the protein substrate are applied using a bioprinter in such a way as the cells are in a concentrated suspension, the volume of which does not exceed 10 μl per cm² of the scaffold.

20. The method according to the claim 4, characterized in that after step (e), the scaffold with cells in the form of a disc is cut out with a cutter.
